(19) United States
(12) Patent Application Publication
(10) Pub. No.: US 2025/0295934 A1
(43) Pub. Date: Sep. 25, 2025

(54) LASER LIGHT-EMITTING DEVICE FOR IRRADIATING SCALP

(71) Applicant: YU-WEN TUNG, Taoyuan City (TW)

(72) Inventor: YU-WEN TUNG, Taoyuan City (TW)

(21) Appl. No.: 18/951,663

(22) Filed: Nov. 19, 2024

(30) Foreign Application Priority Data
Mar. 25, 2024 (TW) 113202932

(51) Int. Cl. A61N 5/06; A61N 5/067

(52) U.S. Cl. CPC A61N 5/0617; A61N 5/067; A61N 2005/0632; A61N 2005/0659; A61N 2005/0663

(57) ABSTRACT

A laser light-emitting device for irradiating the scalp includes a main body, laser modules, and a control member. The main body is in a curved shape and has an inner surface and an outer surface. The inner surface and the outer surface are respectively located on two opposite sides of the main body, a light-emitting area is defined on the inner surface of the main body, and the light-emitting area does not include protrusions. The laser modules are surface mount device components. The laser modules are disposed on the main body, and correspond to the light-emitting area. The control member is disposed on the main body. The laser modules are electrically connected to the control member, and the control member is capable of driving the plurality of laser modules, such that the plurality of laser modules emit laser light from the inner surface of the main body.

### CROSS-REFERENCE TO RELATED PATENT APPLICATION

This application claims the benefit of priority to Taiwan Patent Application No. 113202932, filed on Mar. 25, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a laser light-emitting device for irradiating the scalp, and more particularly to a laser light-emitting device that can be worn on a head of a user and can emit laser light to irradiate the scalp of the user to stimulate hair follicles and facilitate hair growth.

### BACKGROUND OF THE DISCLOSURE

Baldness and hair loss has become an issue for most people. With the advancement of medical technology, more and more treatments are able to treat baldness and hair loss. Clinically, the treatments can be generally divided into two categories of hair growth treatments and hair transplant surgeries. For the hair growth treatments, in addition to hair tonics and oral-administered medications, various physical therapies and laser hair rejuvenation treatments are also available. Specifically, the laser hair rejuvenation treatment has become a widely used treatment in recent years, capable of effectively alleviating the problem of baldness and hair loss.

An existing laser hair growth device can adopt helmet-type or comb-type designs. A comb-type laser hair growth device includes multiple comb teeth and multiple laser modules, the comb teeth can be used to separate hair, and the laser modules can emit laser light and irradiate the scalp of a user to stimulate hair follicles, thereby facilitating hair growth. However, in existing comb-type laser hair growth devices, the comb teeth interfere with the irradiation of laser light, causing the light output of the laser module to be blocked, thus affecting the irradiation efficiency of the laser module and reducing the treatment effect. Furthermore, the configurations of the comb teeth increase the distance between the laser module and the scalp, and will also affect the irradiation efficiency of the laser module, thus reducing the treatment effect.

Furthermore, the laser modules of existing laser hair growth devices usually adopt a traditional can (transistor outline-can, TO-CAN) packaging, which has slow processing speed, high processing cost, large volume, and heavy weight, thus causing inconvenience during production.

### SUMMARY OF THE DISCLOSURE

In response to the above-referenced technical inadequacies, the present disclosure provides a laser light-emitting device for irradiating the scalp. The laser light-emitting device can be used to irradiate the scalp of the user to stimulate the hair follicles and facilitate hair growth. The light-emitting area adopts a design without comb teeth, such that the laser light emission is not interfered with, and the irradiation distance of the laser light can be shortened to improve the irradiation efficiency.

In response to the above-referenced technical inadequacies, the present disclosure further provides a laser light-emitting device for irradiating the scalp. The laser modules are surface mount device (SMD) components, which have advantages such as fast processing speed, low processing cost, small size, light-weight, and convenience of use.

In order to solve the above-mentioned problems, one of the technical aspects adopted by the present disclosure is to provide a laser light-emitting device for irradiating the scalp. The laser light-emitting device includes a main body, a plurality of laser modules, and a control member. The main body is in a curved shape and has an inner surface and an outer surface. The inner surface and the outer surface are respectively located on two opposite sides of the main body, a light-emitting area is defined on the inner surface of the main body, and the light-emitting area does not include protrusions. The plurality of laser modules are surface mount device (SMD) components. The plurality of laser modules are disposed on the main body, and the plurality of laser modules correspond to the light-emitting area. The control member is disposed on the main body. The plurality of laser modules are electrically connected to the control member, and the control member is capable of driving the plurality of laser modules, such that the plurality of laser modules emit laser light from the inner surface of the main body.

At least one light-homogenizing element can be disposed on the main body, such that the laser light generated by the laser modules can be diffused and emitted outward through the light-homogenizing element. Furthermore, the laser light generated by the laser modules can irradiate uniformly, and an irradiation area can be increased.

Therefore, in the laser light-emitting device for irradiating the scalp provided by the present disclosure, the laser light-emitting device includes a main body, a plurality of laser modules, and a control member. The main body is in a curved shape, a light-emitting area is defined on the inner surface of the main body, and the light-emitting area does not include protrusions. The plurality of laser modules are disposed on the main body, and the plurality of laser modules correspond to the light-emitting area. The control member can drive the plurality of laser modules, such that the plurality of laser modules emit laser light from the inner surface of the main body. The laser light can be used to irradiate that scalp of the user to stimulate hair follicles and facilitate hair growth. The light-emitting area of the present disclosure is designed without protrusions (i.e., comb teeth), such that the emission of the laser light is not interfered with. Therefore, the laser light-emitting device of the present disclosure can have better irradiation efficiency and improved treatment effect. Furthermore, the light-emitting area does not include protrusions, such that an irradiation distance of the laser light is shortened, thereby increasing the irradiation efficiency and improving the treatment effect. Moreover, the laser modules are SMD components that have advantages such as fast processing speed, low processing cost, small size, light-weight, and convenience of use.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a perspective view of a laser light-emitting device for irradiating the scalp according to a first embodiment of the present disclosure;

FIG. 2 is another perspective view of the laser light-emitting device for irradiating the scalp according to the first embodiment of the present disclosure;

FIG. 3 is a front view of the laser light-emitting device for irradiating the scalp according to the first embodiment of the present disclosure;

FIG. 4 is a bottom view of the laser light-emitting device for irradiating the scalp according to the first embodiment of the present disclosure;

FIG. 5 is a perspective view of a laser light-emitting device for irradiating the scalp according to a second embodiment of the present disclosure;

FIG. 6 is a front view of a laser light-emitting device for irradiating the scalp according to a third embodiment of the present disclosure;

FIG. 7 is a front view of a folded state of the laser light-emitting device for irradiating the scalp according to the third embodiment of the present disclosure;

FIG. 8 is a perspective view of a laser light-emitting device for irradiating the scalp according to a fourth embodiment of the present disclosure;

FIG. 9 is a perspective view of a laser light-emitting device for irradiating the scalp according to a fifth embodiment of the present disclosure;

FIG. 10 is a front view of a laser light-emitting device for irradiating the scalp according to a sixth embodiment of the present disclosure; and

FIG. 11 is a perspective view of a laser light-emitting device for irradiating the scalp according to a seventh embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

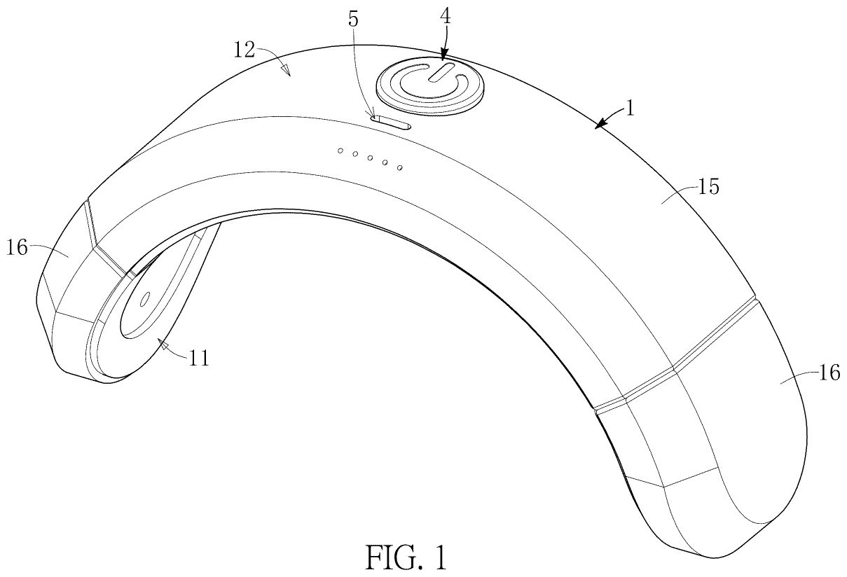

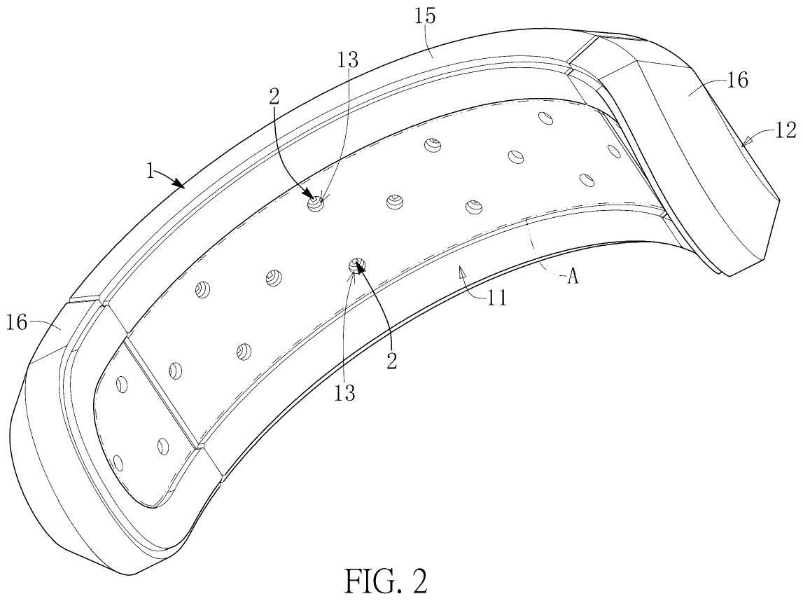

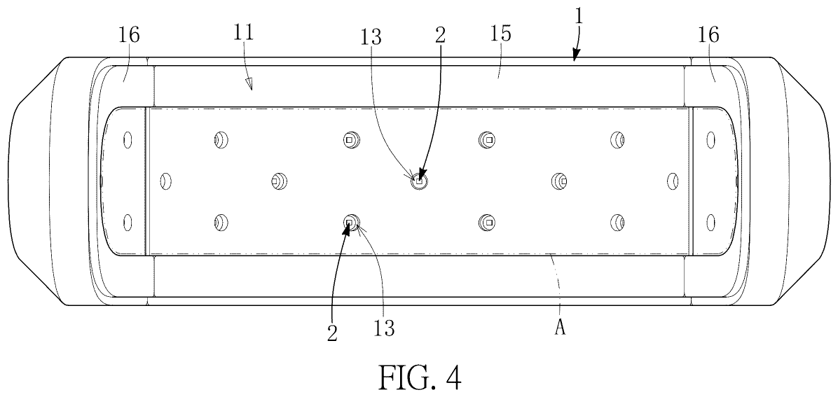

Referring to FIG. 1 to FIG. 4, the present disclosure provides a laser light-emitting device for irradiating the scalp, and the laser light-emitting device includes a main body 1, a plurality of laser modules 2, and a control member 3.

The main body 1 is in a curved shape, and the main body 1 is bent along a length direction of the main body 1. A bottom side and front and rear sides of the main body 1 form openings for being worn on the head of a user, and the main body 1 can be clamped and positioned on the head of the user by utilizing the elasticity of the main body 1. The main body 1 can be made of plastic or other materials, and the present disclosure is not limited thereto. The main body 1 can also be made in a variety of sizes to suit different users. The main body 1 has an inner surface 11 and an outer surface 12. The inner surface 11 and the outer surface 12 are respectively located on two opposite sides of the main body 1. The main body 1 can be worn on the head of the user, such that the inner surface 11 of the main body 1 faces the head (i.e., the scalp) of the user. A light-emitting area A is defined on the inner surface 11 of the main body 1, and the laser modules 2 can be disposed on the light-emitting area A to form an irradiation area of laser light.

In this embodiment, a completely comb-teeth-free design is adopted, and the inner surface 11 of the main body 1 does not include comb teeth, that is, the inner surface 11 of the main body 1 does not include protrusions. Since the inner surface 11 of the main body 1 including the light-emitting area A does not include any protrusions such as comb teeth, the light-emitting area A can be as close to the scalp of the user as possible, and since there are no interference objects between the light-emitting area A and the scalp of the user, a preferred irradiation effect can be obtained.

When a quantity of the laser modules 2 is reduced, a processing cost is also decreased. The plurality of laser modules 2 are disposed on the main body 1, and the plurality of laser modules 2 can be disposed inside of the main body 1, such that the plurality of laser modules 2 do not protrude from the inner surface 11 of the main body 1 and the laser modules 2 have better protection. The plurality of laser modules 2 correspond to the light-emitting area A, and the plurality of laser modules 2 are SMD components. The control member 3 is disposed on the main body 1 and includes a control circuit. Since the control member 3 is an existing technology, details thereof will not be described in detail herein.

The plurality of laser modules 2 are electrically connected to the control member 3. The control member 3 can be used to drive the plurality of laser modules 2 such that the plurality of laser modules 2 emit lasers. The plurality of laser modules 2 can emit laser light from the light-emitting area A of the main body 1. In this embodiment, a plurality of light outlets 13 are formed on the light-emitting area A of the main body 1. The light outlets 13 are arranged at intervals, and the light outlets 13 are distributed in the light-emitting area A. The plurality of laser modules 2 respectively correspond to the plurality of light outlets 13, such that the laser modules 2 can respectively emit laser light outward through the light outlets 13.

In this embodiment, some or all of the laser modules 2 is a multi-wavelength laser device which can be used to emit laser light of multiple wavelengths. These laser modules 2 can emit two, three, four, or five laser beams of equal wavelengths. In this embodiment, the laser modules 2 can emit laser light of various wavelengths between 600 nm and 1550 nm. For example, the laser modules 2 can emit laser light of at least two wavelengths of 600 nm, 650 nm, 660 nm, 780 nm, 808 nm, 830 nm, 905 nm, 940 nm, 950 nm, 980 nm, and 1550 nm. Since the laser module 2 of this embodiment is a multi-wavelength laser device that can be used to emit laser light of various wavelengths, and the laser light of different wavelengths has different penetration depths, the laser light can irradiate hair follicles at various depths for better effect of hair growth.

In this embodiment, a switch 4 and an electrical connector 5 are disposed on the main body 1. The switch 4 and the electrical connector 5 can be disposed on the outer surface 12 of the main body 1. Preferably, the switch 4 and the electrical connector 5 are arranged in the center of the outer surface 12 of the main body 1, such that two ends (left and right ends) of the main body 1 can share a mold to reduce production costs. The switch 4 and the electrical connector 5 are electrically connected to the control member 3. The switch 4 can be used to control the turning-on and turning-off of the laser light-emitting device. The electrical connector 5 can be used to input the required power. In addition, a battery or a rechargeable battery can be provided at the control member 3 to provide the required power.

The laser light-emitting device of the present disclosure can be worn on the head of the user through the main body 1, and drives a plurality of laser modules 2 to emit laser light that is used to irradiate the scalp of the user, so as to stimulate hair follicles and facilitate hair growth.

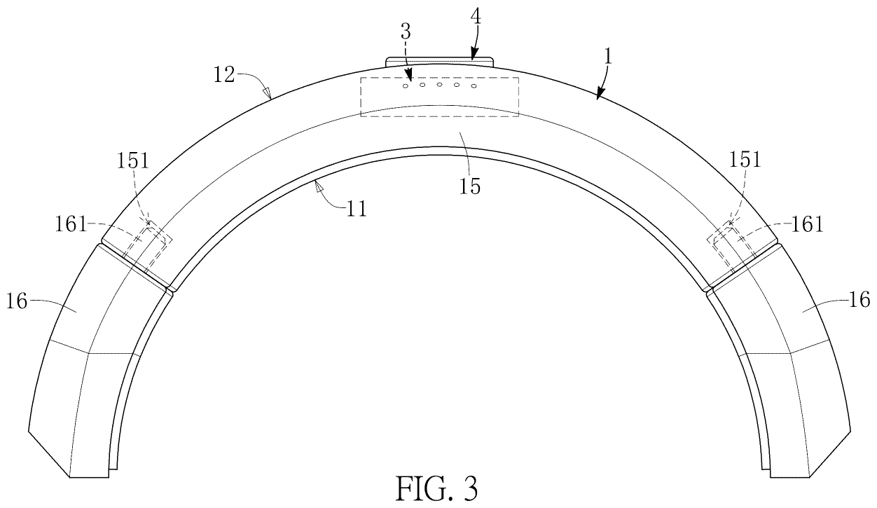

The main body 1 can also be divided into a middle section 15 and two side sections 16. The two side sections 16 can be assembled at two ends of the middle section 15 by insertion. That is, each of the two ends of the middle section 15 respectively includes a first insertion portion 151 (as shown in FIG. 3), each of the two side sections 16 includes a second insertion portion 161. The first insertion portion 151 and the second insertion portion 161 can be corresponding insertion plugs and insertion sockets. The second insertion portions 161 of the two side sections 16 can plug into the first insertion portions 151 at two ends of the middle section 15, such that the middle section 15 and the two side sections 16 have a detachable design for volume reduction and ease of storage. Various conventional connection devices (not shown in the figures) may also be provided between the middle section 15 and the two side sections 16 for transmitting power.

### Second Embodiment

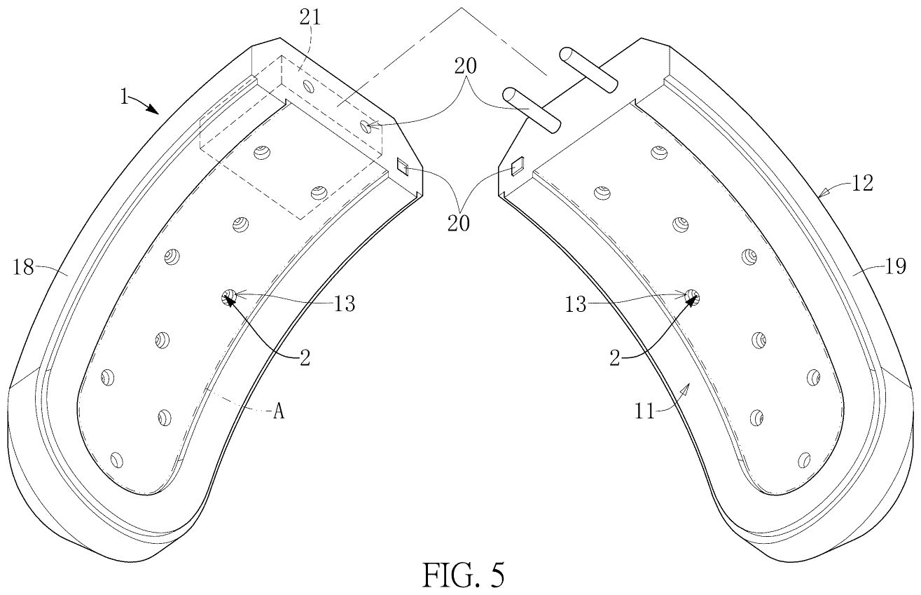

Referring to FIG. 5, in this embodiment, the main body 1 can be divided into two sections, including a first section 18 and a second section 19. The first section 18 and the second section 19 can be detached and separated for volume reduction and ease of storage. A connecting device 20 is provided between the first section 18 and the second section 19. The connecting device 20 can include a plug and a socket, and can be used to connect the first section 18 and the second section 19. The connecting device 20 can also be an electrical connector, and the electrical connector can be such as a conductive terminal, a conductive sheet, or a conductive pad that can have the effect of transmitting electrical power. The connecting device 20 can also be electrically connected with a battery 21 for power supply.

### Third Embodiment

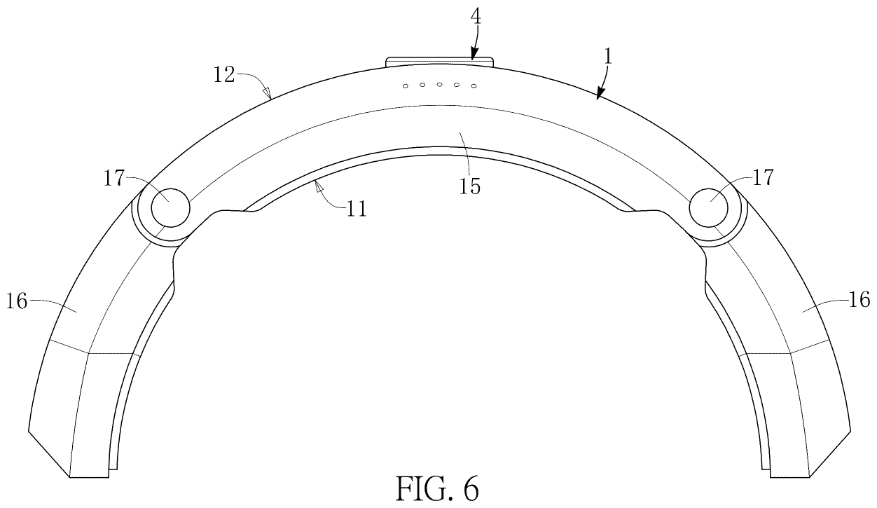

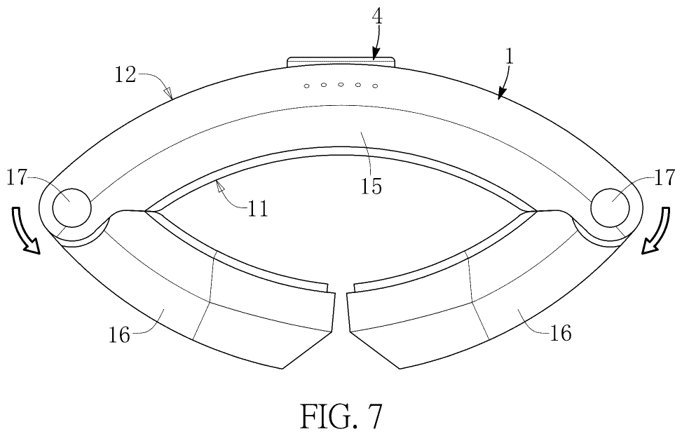

Referring to FIG. 6 and FIG. 7, in this embodiment, the main body 1 can accordingly be divided into the middle section 15 and the two side sections 16. The two side sections 16 are respectively pivotally connected to the two ends of the middle section 15. The two side sections 16 can be respectively pivotally connected to the two ends of the middle section 15 through pivoting mechanisms 17, such that the two side sections 16 can be flipped and folded relative to the middle section 15 (as shown in FIG. 7) for volume reduction and ease of storage.

### Fourth Embodiment

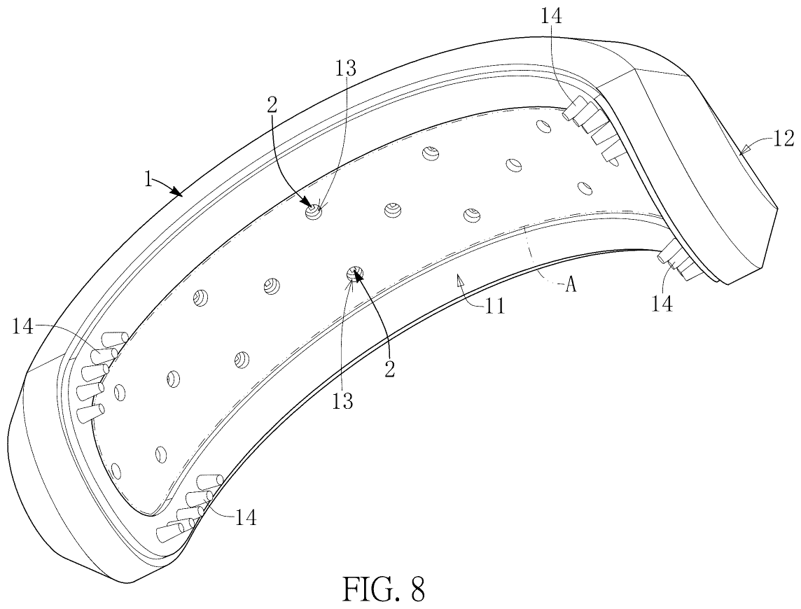

Referring to FIG. 8, in this embodiment, the main body 1 is a one-piece structure, and the main body 1 cannot be disassembled or folded. Furthermore, a plurality of comb teeth 14 are provided on the inner surface 11 of the main body 1. Each of the plurality of comb teeth 14 is column-shaped; preferably, each of the plurality of comb teeth 14 is cylindrical, and can also be tapered. The plurality of comb teeth 14 are arranged outside of the light-emitting area A. The plurality of comb teeth 14 can be arranged adjacent to left and right ends of the inner surface 11 of the main body 1. That is, the plurality of comb teeth 14 are adjacent to two short sides of the main body 1. The plurality of comb teeth 14 are disposed without affecting the irradiation of the laser modules 2. The light-emitting area A does not have any comb teeth 14, such that the light-emitting area A can be as close to the scalp of the user as possible. No interference is between the light-emitting area A and the scalp of the user.

### Fifth Embodiment

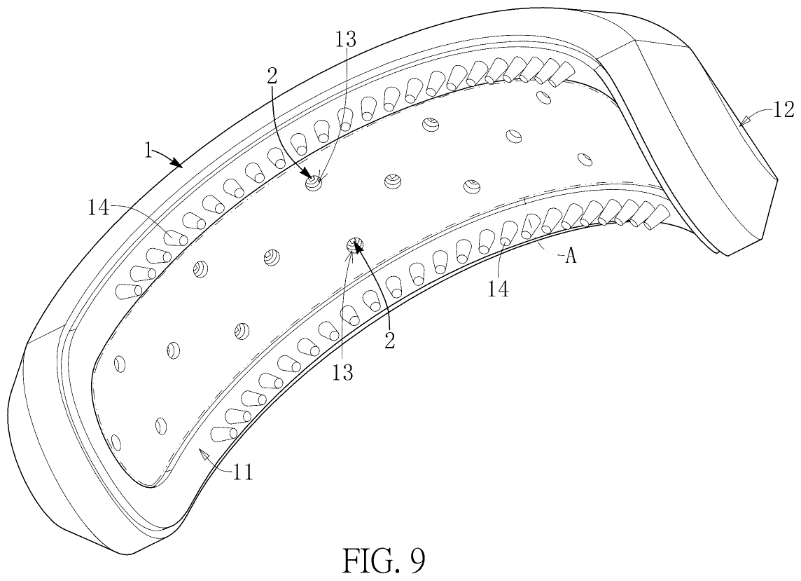

Referring to FIG. 9, in this embodiment, the main body 1 is a one-piece structure, and the main body 1 cannot be disassembled or folded. Furthermore, a plurality of comb teeth 14 are provided on the inner surface 11 of the main body 1. Each of the plurality of comb teeth 14 is column-shaped; preferably, each of the plurality of comb teeth 14 is cylindrical, and can also be tapered. The plurality of comb teeth 14 are arranged outside of the light-emitting area A. The plurality of comb teeth 14 can be arranged adjacent to front and rear sides of the inner surface 11 of the main body 1. That is, the plurality of comb teeth 14 are adjacent to two long sides of the main body 1. The plurality of comb teeth 14 are disposed without affecting the irradiation of the laser modules 2. The light-emitting area A does not have any comb teeth 14, such that the light-emitting area A can be as close to the scalp of the user as possible. No interference is between the light-emitting area A and the scalp of the user.

### Sixth Embodiment

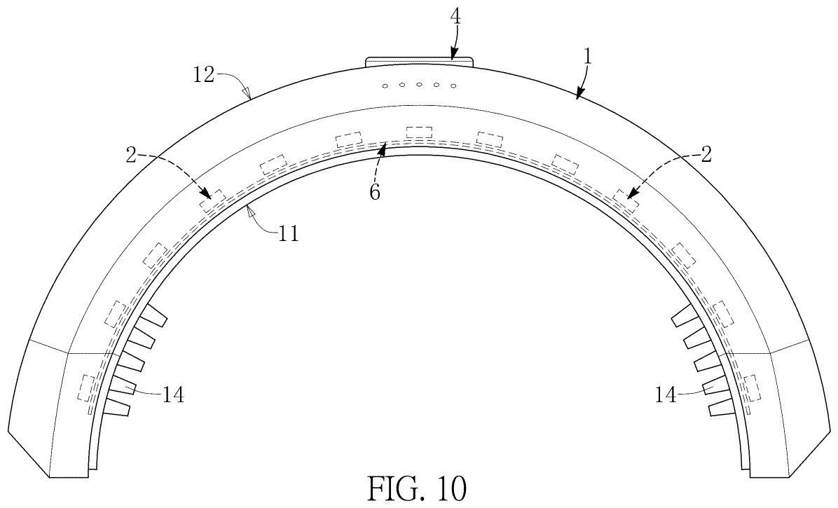

Referring to FIG. 10, in this embodiment, at least one light-homogenizing element 6 is disposed on the main body 1, such that the laser light generated by the laser modules 2 can be diffused and emitted outward through the light-homogenizing element 6. Furthermore, the laser light generated by the laser modules 2 can irradiate uniformly, and irradiation area can be increased.

### Seventh Embodiment

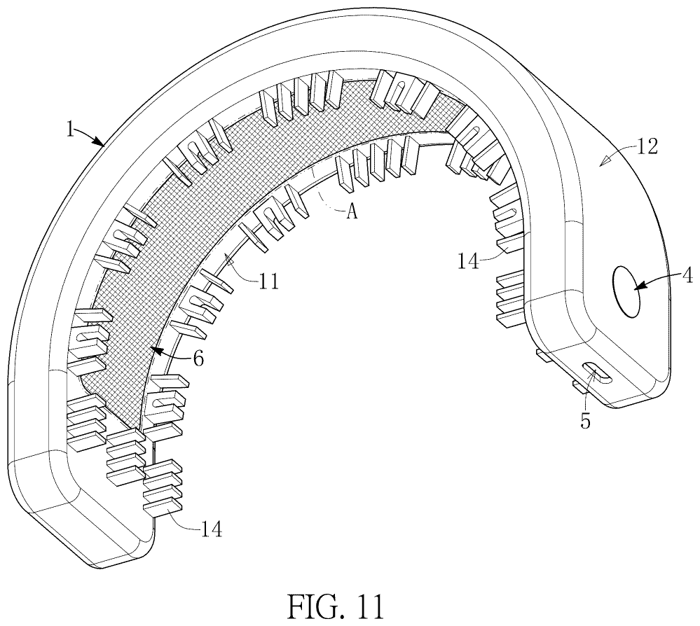

Referring to FIG. 11, in this embodiment, the main body 1 is a one-piece structure, and the main body 1 cannot be disassembled or folded. Furthermore, a plurality of comb teeth 14 are provided on the inner surface 11 of the main body 1. Each of the plurality of comb teeth 14 is column-shaped; preferably, each of the plurality of comb teeth 14 is a rectangular column. The plurality of comb teeth 14 are arranged outside of the light-emitting area A. The plurality of comb teeth 14 are disposed without affecting the irradiation of the laser modules 2. The light-emitting area A does not have any comb teeth 14, such that the light-emitting area A can be as close to the scalp of the user as possible. No interference is between the light-emitting area A and the scalp of the user.

### Beneficial Effects of the Embodiments

In conclusion, in the laser light-emitting device for irradiating the scalp provided by the present disclosure, the laser light-emitting device includes a main body, a plurality of laser modules, and a control member. The main body is in a curved shape, a light-emitting area is defined on the inner surface of the main body, and the light-emitting area does not include protrusions. The plurality of laser modules are disposed on the main body, and the plurality of laser modules correspond to the light-emitting area. The control member can drive the plurality of laser modules, such that the plurality of laser modules emit laser light from the inner surface of the main body. The laser light can be used to irradiate the scalp of the user to stimulate hair follicles and facilitate hair growth. The light-emitting area of the present disclosure is designed without protrusions (i.e., comb teeth), such that the emission of the laser light is not interfered with. Therefore, the laser light-emitting device of the present disclosure can have better irradiation efficiency and improved treatment effect. Furthermore, the light-emitting area does not include protrusions, such that an irradiation distance of the laser light is shortened, thereby increasing the irradiation efficiency and improving the treatment effect. Moreover, the laser modules are SMD components that have advantages such as fast processing speed, low processing cost, small size, light-weight, and convenience of use.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A laser light-emitting device for irradiating the scalp, comprising:
a main body being in a curved shape and having an inner surface and an outer surface, wherein the inner surface and the outer surface are respectively located on two opposite sides of the main body, a light-emitting area is defined on the inner surface of the main body, and the light-emitting area does not include protrusions;
a plurality of laser modules being surface mount device (SMD) components, wherein the plurality of laser modules are disposed on the main body, and the plurality of laser modules correspond to the light-emitting area; and
a control member disposed on the main body, wherein the plurality of laser modules are electrically connected to the control member, and the control member is capable of driving the plurality of laser modules, such that the plurality of laser modules emit laser light from the inner surface of the main body.

2. The laser light-emitting device according to claim 1, wherein a plurality of light outlets are formed on the inner surface of the main body, the plurality of light outlets are arranged at intervals, and the plurality of light outlets are distributed in the light-emitting area; wherein the plurality of laser modules respectively correspond to the plurality of light outlets.

3. The laser light-emitting device according to claim 1, wherein a portion or all of the laser modules are multi-wavelength laser devices that are able to emit laser light of multiple wavelengths, and each of the laser modules is able to emit laser light of various wavelengths between 600 nm and 1550 nm.

4. The laser light-emitting device according to claim 1, wherein a switch and an electrical connector are disposed on the main body, the switch and the electrical connector are arranged on the outer surface of the main body, and the switch and the electrical connector are electrically connected to the control member.

5. The laser light-emitting device according to claim 1, wherein a plurality of comb teeth are disposed on the inner surface of the main body, each of the plurality of comb teeth is column-shaped, and the plurality of comb teeth are arranged outside of the light-emitting area; wherein the plurality of comb teeth are arranged on the inner surface of the main body adjacent to left and right ends or front and rear sides of the inner surface.

6. The laser light-emitting device according to claim 1, wherein at least one light-homogenizing element is disposed on the main body, and the laser light generated by the plurality of laser modules is able to be diffused and emitted outward through the light-homogenizing element.

7. The laser light-emitting device according to claim 1, wherein the main body is divided into a middle section and two side sections; wherein the two side sections are respectively pivotally connected to two ends of the middle section, and the two side sections are able to be flipped and folded relative to the middle section.

8. The laser light-emitting device according to claim 1, wherein the main body is divided into a middle section and two side sections, and the two side sections are assembled by insertion at two ends of the middle section, such that the middle section and the two side sections have a detachable design.

9. The laser light-emitting device according to claim 1, wherein the inner surface of the main body does not include protrusions.

10. The laser light-emitting device according to claim 1, wherein the main body includes a first section and a second section, the first section and the second section are able to be detached and separated; wherein a connecting device is provided between the first section and the second section for connecting the first section and the second section, and the connecting device is an electrical connector.
